(19) United States
(12) Patent Application Publication
(10) Pub. No.: US 2004/0002721 A1
(43) Pub. Date: Jan. 1, 2004

(54) Method and apparatus for performing end-to-end and end-to-side anastomosis with eversion of tissue edges

(76) Inventors: Jonathan L. Podmore, San Francisco, CA (US); Michael Francis Wei, San Mateo, CA (US); Timothy J. McCoy, San Carlos, CA (US); Timothy B. McFann, Redwood City, CA (US); Peter Callas, Redwood City, CA (US); Geoffrey H. Willis, Rancho Cucamonga, CA (US); Paul A. Spence, Louisville, KY (US)

Correspondence Address: Alan W. Cannon, Law Office of Alan W. Cannon, 834 South Wolfe Road, Sunnyvale, CA, 94086, US

(21) Appl. No.: 10/374,350

(22) Filed: Feb. 25, 2003

Related U.S. Application Data
(63) Continuation of application No. 09/583,944, filed on May 31, 2000, now abandoned.
(60) Provisional application No. 60/152,001, filed on Sep. 1, 1999.

(51) Int. Cl. A61B 17/08

(52) U.S. Cl. 606/155

(57) ABSTRACT

An element for use in anastomosis and a method and apparatus for installing the element (having tines that extend from a central portion) at the open end of a blood vessel (or other tubular body structure). To install an embodiment of the element; a carriage holds the element and shields the tines, the vessel's open end is passed through the shielded element and fitted over an anvil, the tines are exposed, and the carriage is actuated to fire the element against the anvil so that the tines pierce the vessel and curl against the anvil (everting the tissue around the vessel's open end). The anvil preferably forms the vessel end portion into a hooded shape suitable for creating an anastomotic junction. Or, the element is installed by inserting an intraluminal anvil through an incision in the vessel's upper wall, advancing an extracorporeal anvil against the vessel's lower wall (pinching the vessel closed at a location between the incision and the vessel's open end), and firing the element against the anvils so that tines at the incision's heel pierce the upper wall, tines at the incision's toe pierce both upper and lower wall, and all tines curl against the anvils. Preferably, the element is formed from sheet metal with the tines extending out from the central portion's inner edge and the tines are then bent relative to the central portion. Alternatively, the element is installed by sliding it onto (and inserting an anvil in) the vessel and folding it to cause the tines to pierce the vessel and curl against the anvil, or the element is C-shaped and is installed by sliding it onto the vessel, squeezing it to make it O-shaped, and firing it against an anvil that has been inserted in the vessel.

### CROSS-REFERENCE TO RELATED APPLICATION

This patent application is a non-provisional application filed under 35 U.S.C. §111(a) claiming priority under 35 U.S.C. § 119(e)(l) based on provisional application No. 60/152,001, filed Sep. 1, 1999, the full text of which provisional application is incorporated herein by reference.

### TECHNICAL FIELD OF THE INVENTION

The present invention relates to the art of surgery. More specifically, it relates to the field of apparatus and methods for performing anastomosis without hand-suturing.

### BACKGROUND OF THE INVENTION

In the United States, many coronary artery bypass graft (CABG) procedures are performed on patients annually. Each of these procedures may include one or more graft vessels which are typically hand sutured. Until recently, coronary artery bypass procedures have been performed with the patients on cardiopulmonary bypass while the heart is arrested with cardioplegia and the surgery is performed on an exposed, stationary heart.

The vast majority of CABG procedures performed currently are accomplished by opening the chest wall to gain access to the coronary vessels. Through the use of heart lung bypass machines and a drug to protect the heart muscle, the heart is stopped and remains still during the procedure. In this setting, the surgeon has ample time and access to the vessels to manipulate hand suturing instruments such as forceps, needle holders and retractors.

However, with increasing costs of hospital stays and increased awareness by patients of other minimally invasive surgical procedures, interest in developing a minimally invasive CABG procedure is increasing. Hospitals need to reduce costs of procedures and patients would like less post-operative pain and speedier recovery times.

With an increased incentive to reduce costs, there is a renewed interest in redesigning cardiothoracic procedures. A few pioneering surgeons are now performing minimally invasive procedures whereby the coronary artery bypass is performed through a small incision in the chest wall. There are some surgeons that believe that the best way to perform a minimally invasive coronary artery bypass procedure is to perform the procedure on a beating heart, i.e., without heart-lung bypass and cardioplegia. This minimizes the time it takes to perform the procedure and reduces the cost of the operation by eliminating the heart lung bypass machine.

In the case of minimally invasive procedures on a beating heart, the surgeon starts by making a mini-thoracotomy between the fourth and fifth ribs and, sometimes, removing the sternal cartilage between the fourth or fifth rib and the sternum. The space between the fourth and fifth ribs is then spread to gain access to the internal mammary artery (IMA) which is dissected from the wall of the chest. After dissection, it is used as the blood supply graft to the left anterior descending artery of the heart (LAD) or other diseased artery. Below the IMA lies the pericardium and the heart. The pericardium is opened exposing the heart. At this point, the LAD may be exposed, or dissected from the fissure of the heart and suspended up with soft ligatures to isolate the artery from the beating heart. Typically, a special retractor gently applies pressure to the heart muscle to dampen movement at the diseased coronary vessel. A small arteriotomy is performed in the diseased vessel and the graft IMA is sutured thereto.

Traditionally, to gain access to the cardiac vessels to perform this procedure the sternum is sawn in half and the chest wall is separated. Although this procedure is well perfected the patient suffers pain and a long recovery. Also, standards for less invasive CABG procedures involve a beating heart without cardioplegia, accessed through a sternotomy.

Until recently all bypass graft procedures have been performed by hand suturing the tiny vessels together with extremely fine sutures under magnification. The skills and instruments required to sew extremely thin fragile vessel walls together have been perfected over the last twenty years and are well known to the surgical community that performs these procedures.

In some conventional anastomoses using hand-sutures, a coronary artery and graft vessel are connected in a side-to-side fashion. One end of the graft vessel is tied closed, and the side wall of the graft vessel near this closed end is hand-sutured to the artery (at a “distal” graft site). At the distal graft site, an incision is made in the artery and a corresponding incision is made in the graft vessel, the incisions are aligned, and the edges of the aligned incisions are hand-sutured together to connect the artery to one end of the graft vessel. The opposite end of the graft vessel is hand-sutured to the aorta (at a “proximal” graft site). Hand-suturing can also be used to perform an end-to-side anastomosis, in which an open end of a graft vessel is aligned with an incision in the sidewall of another vessel (e.g., an aorta) and the aligned tissue is hand-sutured together. The present invention can be used to perform either end-to-side or end-to-end anastomosis without hand-suturing.

There is a need (addressed by the present invention) for methods and apparatus useful for performing anastomosis during CABG surgery on a beating heart. When performing anastomosis during such surgery on a beating heart, use of hand-suturing to attach the graft vessel is very imprecise due to the translation of movement from the beating heart to the suspended artery. This motion may cause imprecise placement of the suture needles. Any imprecise placement of the sutures may cause a distortion of the anastomosis which may cause stenosis or leaks at this junction. The sutures used for this procedure are extremely fine (0.001″ in diameter) and are placed less than 1 mm apart.

As one can imagine it is difficult enough to place suture needles the size of a small eyelash into a vessel wall with placement accuracy of better than 1 mm. To accomplish this feat of precision on a moving target is extremely difficult. To make matters worse, the site is often bloody due to the fact that the heart has not been stopped. During beating heart surgery, the surgeon can attempt to minimize the deleterious effects of the beating heart motion by using suspension or retraction techniques, but it is impossible to isolate all such movement (and attempts to minimize the motion can damage the vessel being restrained or cause myocardial injury).

Even when performing anastomoses in an ‘open chest’ surgical setting in which the surgeon has adequate access and vision of the surgical site to manipulate the anatomy and instruments, it is difficult to perform the hand-suturing required in traditional methods. When performing anastomoses in a minimally invasive procedure, access to (and vision of) the site is more limited and the hand-suturing is more difficult.

If the sutures are not placed correctly in the vessel walls, bunching or leaks will occur. During a minimally invasive procedure this is disastrous, usually resulting in the conversion to an open chest procedure to correct the mistake. Any rough handling of the vessel walls is detrimental as inflammation can cause further postoperative complications.

An anastomosis must seal without leaking to prevent exsanguination. Therefore, any anastomosis technique which does not require hand sutures must provide a leak free seal in a very confined space, while providing proper flow area in the vessel after healing is complete.

Although minimally invasive CABG procedures are taking place now with hand-sutured anastomosis they require superlative surgical skills and are therefore not widely practiced. There is a need for methods and apparatus which permit the forming of a precise anastomosis without requiring the stopping of a beating heart, during either minimally invasive or open chest surgery, and without requiring hand suturing.

Several techniques have been proposed for performing anastomosis of blood vessels. However, the prior art techniques often require the vessels to be severely deformed during the procedure. The deformation may be required to fit the vessels together or to fit a vessel to an anchoring device.

For example, some prior art anastomosis techniques have used rigid rings to join two vessels together. In one such technique (to be described with reference to FIG. 1), rigid ring 30′ is positioned around the edges of an incision in the sidewall of artery 31 in a manner that inverts the tissue near the incised edges, in the sense that the tissue is everted to expose the inside lining (intima) of the vessel walls. The incised edges can be anchored on a flange (not shown) on ring 30′. Rigid ring 30″ is positioned around the open end of graft vessel 31 in a manner that inverts the tissue at the open end (by everting the tissue), thereby exposing the intima of vessel 31. Then, rings 30′ and 30″ are moved into alignment with each other and fastened together (e.g., by a clamp) so that the intima of the vessels are clamped together in contact with each other.

In another such technique (to be described with reference to FIG. 2), rigid ring 30 is positioned around the open end of vessel 33 in a manner that inverts the tissue at the open end (by everting the tissue), thereby exposing the intima of vessel 33. Then, the open end of vessel 34 is fitted over (and fastened to) the ring-containing end of vessel 33.

However, it is undesirable to simply slit side-wall tissue of a vessel and pull the incised edges through a ring (as in FIG. 1) to anchor them on a flange (or to invert and pull tissue at the end of a vessel over a ring as in FIG. 2). Pulling or stretching the vessel walls produces a very unpleasant and unexpected result. Vessel walls are made of tissue fibers that run in the radial direction in one layer and the longitudinal direction in another layer. The elasticity of the tissue fibers in the longitudinal direction is greater than those that run radially. Therefore, the tissue will not stretch as easily in the radial or circumferential direction and results in a narrowing or restriction when pulled or stretched in the prior art devices. Also, vessels can spasm if treated harshly. Manhandling will result in restrictions and stenotic junctions because the vessel walls will react poorly to being treated in a rough manner and the stretching of the vessel wall will telegraph up the vessel wall due to the high radial stiffness of the vessel structure, causing restrictions and spasms in the vessel wall.

Additionally, prior art methods and apparatus for anastomosis without hand-suturing do not adequately ensure hemostasis to avoid leakage from the anastomosis junction under pressure, and they attempt to accomplish hemostasis through excessive clamping forces between clamping surfaces or stretching across over-sized fittings.

In order to effect good healing, healthy vessel walls must be brought into intimate approximation. This intimate approximation can be accomplished by the skilled hands of a surgeon with sutures. A vascular surgeon is taught how to suture by bringing the vessel edges together with just the right knot tightness. If the edges are tied too loosely, the wound will leak and have trouble healing causing excessive scar tissue to form. If the edges are tied too tightly, the sutures will tear through the delicate tissue at the suture hole causing leaks. The key is to bring the edges together with just the right amount of intimate approximation without excessive compression.

Conventional junctions that include rings are anatomically incorrect both for blood flow and for healing. A well made anastomotic junction is not made in a single plane and should accurately follow blood vessel geometry. The junction is more of a saddle shape, and the cross section is not necessarily a circle. The junction where the vessel units join is not a constant cross section angle, but an angle that varies continuously throughout with respect to any linear reference. In addition, the length of the junction should be many times the width of the opening in order to assure a low blood flow pressure gradient in the junction and to assure a proper flow area. In fact, the best results are obtained if the confluence area is actually oversized. The prior art junctions do not account for such flow characteristics and parameters and are thus deficient. There is a need for an anastomotic technique which can establish proper flow characteristics and parameters and that accurately preserves blood vessel geometry, specifically the plural planar nature in which the junction occurs. Furthermore, most anastomoses are made between vessels that are not similar in size. It is therefore necessary to provide a means and method which allow for the accommodation and joining of dissimilarly sized vessels.

After attachment of a graft vessel by anastomosis, the supply vessels grow in diameter to accommodate their new role in providing oxygenated blood to the heart. Therefore, there is a need to provide a junction that will accommodate any increase in the dimension of the graft vessel size. With a rigid ring that is a singular circular cross section of the graft, the fitting does not allow the vessel to provide this increase in flow as the vessels expand to meet the needs of the heart muscle. Still further, the inside lining of the vessel walls (intima) should make contact with each other (for a variety of reasons). The walls of the joined vessels must come together with just the right amount of approximation to promote good healing and prevent leakage and formation of false lumens. If the incised edges are too far apart scarring will occur causing restrictions. The walls cannot be compressed tightly between two hard surfaces which will damage the vessels. The prior art teaches plumbing-like fittings clamped onto vascular structures. However, clamping and compressing the vessel walls too tightly will cause necrosis of the vessel between the clamps. If necrosis occurs the dead tissue will become weak and most likely cause a failure of the joint. Still further such rings and tubes used to clamp vessels together do not follow the correct anatomical contours to create an unrestricted anastomosis. Failing to account for the way healing of this type of junction occurs, and not accounting for the actual situation may cause a poor result.

A suture technique has the advantage of having the surgeon making on-the-fly decisions to add an extra suture if needed to stop a leak in the anastomosis. In a mechanical minimally invasive system it will not be possible to put in an ‘extra suture throw’ so the system must provide a way to assure complete hemostasis. Approximation using a mechanical system will not be perfect. If the design errs on the side of not over-compressing the tissue, there may be very small areas that may present a leak between the edges of the vessel walls. Healing with prior art techniques using mechanical joining means is not as efficient as it could be. There is a need for an anastomotic technique that accounts for the way healing actually occurs and provides proper structural support during the healing process.

Conventional means and methods of performing an anastomosis do not permit the formation of multiple anastomotic sites on a single graft vessel such as at both proximal and distal ends. Thus a surgeon will have to use multiple tools to perform multiple anastomoses. This will be either impossible or very expensive. Therefore, there is a need for a means and a method for performing an anastomosis which will lend itself to efficient and cost-effective multiple by-pass techniques.

As noted above, performing anastomosis in a minimally invasive manner while the patient's heart is beating requires an extremely high degree of dexterity. Any apparatus used in such a procedure must therefore be as easy and efficient to use as possible so that a surgeon can focus most of his or her attention on the anastomosis site.

Further, any apparatus used for anastomosis without hand-suturing should be amenable to efficient manufacture.

U.S. Pat. No. 5,868,763, issued Feb. 9, 1999, teaches methods and apparatus for accomplishing anastomosis without hand-suturing in a manner overcoming many of the disadvantages of conventional anastomosis methods and apparatus such as those described above. The apparatus of U.S. Pat. No. 5,868,763 includes a flexible “cuff” having tines configured to pierce a vessel or other organ (e.g., to penetrate tissue around the edges of an incision in the side-wall of a blood vessel) to attach the cuff to the vessel or organ. When deformed, the cuff remains in the deformed configuration until physically moved into another configuration. Various embodiments of the cuff can be mounted to the open end of blood vessel or around an incision in the sidewall of a blood vessel (or other organ), and then deformed to open or close the vessel end or incision as desired, so that various embodiments of the cuff can be used to perform end-to-end anastomosis (in which the open end of one vessel is attached to the open end of another vessel), end-to-side anastomosis (in which the open end of one vessel is attached in fluid communication with an incision in the side wall of another vessel), or side-to-side anastomosis (to attach the side wall of one vessel to the side wall of another vessel).

When implementing side-to-side anastomosis, one cuff is attached around an incision in the side wall of the first vessel and another cuff is typically attached around an incision in the side wall of the other vessel. The cuffs are then aligned and fastened together. However, the cuffs are designed (and attached to the vessels) such that when the two cuffs are aligned, the incised tissue edges of the two vessels are placed in edge-to-edge contact (so that there is a risk that the anastomosis will be completed without the intima of the two vessels being in direct contact with each other at all locations where the vessels meet each other).

U.S. Pat. No. 5,868,763 describes (with reference to FIGS. 35, 36A-36D, and 37 thereof) a tool having an anvil and a translatable housing for installing one of the cuffs at the open end of a blood vessel. The anvil of the installation tool receives the cuff and distal end portion of the vessel, and positions the cuff around the vessel's open end. The housing defines a concave cavity (shaped to conform with a convex portion of the anvil's surface). To install the cuff around the vessel's open end (while forming the vessel's end portion into a shape suitable for producing an anastomosis), the housing is translated into engagement with the cuff. This causes the housing's cavity to push the tips of the cuff's tines through the vessel's sidewall so that the tines curl against the anvil, and presses the vessel against the convex surface of the anvil thereby forming the vessel's end portion into a shape suitable for producing an anastomosis. However, the tines of the cuffs are curled in such a manner that when two installed cuffs are aligned, the incised tissue edges of the two vessels are placed in edge-to-edge contact rather than intima-to-intima contact.

Anastomosis rings designed and installed in accordance with the present invention are useful for performing end-to-end anastomosis or end-to-side anastomosis, with direct and uniform intima-to-intima contact being achieved in both cases.

U.S. Provisional Application 60/152,001, filed Sep. 1, 1999, discloses several types of malleable rings for use in anastomosis, including those shown in FIGS. 3-6 of the present disclosure. Ring 50 (shown in FIGS. 3-5) includes a ring portion 54, and tines 51 and docking arms 52 that extend from the outer edge of ring portion 54. Each docking arm 52 defines a hole 53 for use in aligning ring 50 with another identical ring, and attaching the two aligned rings together. Ring 50 is integrally formed from metal and ring portion 54 and tines 51 are malleable in the sense that once deformed from a first shape into a second shape, they will not relax back into the first shape from the second shape. FIG. 3 shows tines 51 in their initial, straight configuration.

To install ring 50 in a vessel with ring portion 54 extending around an incision, tines 51 pierce the tissue around the incision and are curled against an anvil until tines 51 have the bent configuration shown in FIG. 4. The action of curling the tines everts the tissue near the orifice edges to expose the inside surface of the vessel or organ (so that such exposed inside surface can be joined to tissue of another vessel).

In typical use, ring 50 is installed (as shown in FIG. 5) at the site of an incision in the side wall of a blood vessel having exterior surface 56 and interior surface (inside lining or “intima”) 55. More specifically, the ring is installed with ring portion 54 (not visible in FIG. 5) extending around the incision, and the action of curling the ring's tines during installation everts the incised edges of the orifice to expose the intima 55 of the blood vessel as shown in FIG. 5.

FIG. 6 of the present disclosure is a top elevational view of another ring (described in Provisional Application 60/152,001) for use in performing anastomosis. Anastomosis ring 80 of FIG. 6 is integrally formed from metal, and includes a ring portion 88, and tines 81 and docking arms 82 that extend from the outer edge of ring portion 88. Each docking arm 82 defines a pair of holes 83 (for use in holding ring 80 during installation in a vessel, aligning an installed ring 80 with another identical ring, and/or attaching together the two aligned rings). Ring portion 88, tines 81, and arms 82 are malleable. Ring 80 is shown in the flat configuration in which it will typically be manufactured. Before use, tines 81 would be bent (each about its line of attachment to ring portion 88) by approximately ninety degrees with unique curvature (out of the plane of FIG. 6) relative to the rest of ring 80.

Each arm 82 has very thin cross section, especially at thin portions 84, so as to have good flexibility. Thin portions 82 are designed to deform plastically with very light force during spreading of ring portion 88 (when the ring is installed at an anastomosis site), and when the ends 86 of arms 82 are pulled away from each other with gentle force by docking forceps (during handling and alignment of two installed rings 80).

The end portion 87 of each arm 82 (near end 86) is made of thicker material than are the thin portions 84, since such reinforcement of the end portions aids in the accuracy with which two rings 80 can be angularly aligned during docking.

The shape of docking arms 82 allows convenient alignment and attachment together of two of rings 80 (each installed in a different vessel) at an anastomosis site, using docking forceps and spring or crimp clips. Notches 85 are configured to snag side loops of a spring clip while the spring clip is sprung around two of rings 80 (that have previously aligned with each other) so that the spring clip clamps the rings together.

The anastomosis ring designs disclosed in provisional application 60/152,001 are for installation in an incision in a side wall of a blood vessel (or other cylindrical vessel or organ), using an anvil of the type also disclosed therein. During installation, the tines curl initially radially inward (toward the incision) and continue to curl so as to evert the tissue around the incision (e.g., from the configuration of FIG. 3 to that of FIG. 4). The disclosed attachment of tines to the outer edge of the ring portion allows efficient eversion in this context. However, the inventors have recognized that to install an anastomosis ring at the open end of a cylindrical (or generally cylindrical) vessel in accordance with the present invention, significant advantages result from use of tines that are attached to the inner edge of the ring portion and are curled initially radially outward (away from the center of the ring portion). Also, the inventors have recognized how to manipulate a cylindrical (or generally cylindrical) vessel and a tined anastomosis ring efficiently so as to insert the vessel's open end through the ring's central orifice (in a manner preventing the tines from engaging and sticking the vessel) and then firing the ring against the vessel to cause the tines to pierce the tissue around the vessel's open end and to curl in a manner everting the vessel's open end.

### SUMMARY OF THE INVENTION

In a class of preferred embodiments, the invention is a method and apparatus for installing an anastomosis element (an object for use in anastomosis, having a central portion which at least partially surrounds a central orifice, and deformable tines extending out from the central portion) at the open end of a blood vessel (or other tubular body structure). Herein, the term “deformable” is used in a broad sense to denote malleable, or otherwise plastically deformable, or having a shape memory, or elastic or super-elastic, in the sense that once a deformable tine is deformed from a first shape into a second shape (in accordance with the invention), it will not relax back into the first shape from the second shape without a change in environment (e.g., temperature) or application of force.

In some embodiments, the anastomosis element of the invention has a central ring portion that surrounds (at least partially) the central orifice, and tines (and typically also docking arms) that extend out from the ring portion. The ring portion is generally flat and the tines are oriented perpendicular (or substantially perpendicular) to the ring portion. The tines (and preferably also the ring portion) are deformable, and preferably the docking arms are flexible. In some embodiments, the flexible docking arms are elastic and in other preferred embodiments they are deformable but not elastic.

In some embodiments of the invention, an anastomosis element is installed at the open end of a blood vessel (or other tubular or generally tubular body structure) as follows. The element is held by a hammer (“carriage”) assembly having a shield assembly which shields the tines during positioning of the vessel. Then, the open end of the vessel is passed through the central portion of the shielded element (in the same direction in which the tines extend) and placed against an anvil so that a protruding portion of the anvil extends inside the vessel. The shield assembly is then actuated to expose the tines, and the carriage assembly is actuated to press the element against the anvil, which causes the tines to pierce and penetrate through the vessel wall and to curl (against the anvil) radially outward. The curled tines of the element evert the tissue around the vessel's open end (to expose the intima of the vessel wall), while the force exerted on the vessel by the carriage assembly, element, and anvil gently forms the end portion of the vessel into a shape (preferably a hooded shape) suitable for creating a patent anastomotic junction.

In other embodiments, an anastomosis element is installed at the open end of a blood vessel (or other tubular or generally tulular body structure) as follows. An anvil (mounted at the end of an elongated stem) is advanced distally into the vessel's open end (causing the tissue around the open end to curl over the anvil's outer edge), the element is advanced distally to cause its tines to pierce the tissue around the vessel's open end and curl against the anvil's proximal surface (thereby everting the tissue), and the anvil stem is tilted to reduce the cross-section of the anvil that faces the installed element, and the stem is pulled to retract the anvil from within the vessel.

Another aspect of the invention is an anastomosis element having a central portion (which at least partially surrounds around a central orifice and has an inner edge nearest the central orifice and an outer edge), docking arms extending out from the outer edge of the central portion (away from the central orifice), and tines extending out from the inner edge of the central portion. Preferably, the element is integrally formed from metal, the central portion is generally flat (does not deviate substantially from a plane) during use, and the tines are oriented perpendicular (or substantially perpendicular) to the central portion during use. Preferably, the element is manufactured in a flat configuration (e.g., it is formed by chemically etching a thin sheet of stainless steel, or is stamped from thin sheet metal), and the tines are bent (each about its line of attachment to the central portion) by ninety degrees relative to the central portion. To install the element in the open end of a blood vessel (or other tubular or generally tubular body structure) with the central portion extending around the open end, the tines pierce the tissue around the open end and are curled against an anvil. The action of curling the tines everts the tissue near the open end to expose the inside surface of the body structure (so that such exposed inside surface can be joined to tissue of another body structure).

Another embodiment of the inventive anastomosis element (sometimes referred to as a “bear trap” element embodiment) has tines that extend out from a foldable central portion. To install the element in the open end of a blood vessel (or other tubular or generally tubular body structure) with the central portion extending around the open end, an anvil is inserted in the open end, the element is folded about a central hinge portion (of the central portion) to cause the tines to pierce the vessel side wall and curl against the anvil, the anvil is then removed, and the element is folded about side hinge portions (of the central portion) to evert the tissue around the open end. Optionally, the end of the vessel is trimmed (or cut off) after removal of the anvil to define a new open end, and the element is then folded about the side hinge portions to evert the tissue around the new open end. For installation of the element at the end of a blood vessel, the anvil is preferably cylindrical or generally cylindrical and its surface defines concave tine-receiving pockets.

Other embodiments of the invention are a method and apparatus for installing an anastomosis element using a two-piece composite anvil. The composite anvil comprises an intraluminal anvil (preferably mounted at the end of an anvil stem) and an extracorporeal anvil. Each anvil has a generally flat tine-forming surface having tine-forming pockets. In use, the intraluminal anvil is inserted through an incision in the sidewall of a tubular body structure (e.g., a graft vessel having an open end) into the body structure's interior. The extracorporeal anvil is not inserted into the structure's interior, but is advanced into engagement with the structure's outer sidewall until the tine-forming surfaces of the two anvils are aligned (coplanar in preferred embodiments), thereby pinching closed the structure at a location between the incision and the structure's open end. An anastomosis element (having a toe end nearest the structure's open end, and a heel end) is then advanced toward the anvils until the tines at the heel end pierce the structure's upper wall, the tines at the toe end pierce both the upper and lower wall of the body structure, and all the tines curl radially outward in response to being forced against the tine-forming anvil surfaces. After the tines have curled (thereby everting the tissue around the incision), the intraluminal anvil is removed through the incision and the extracorporeal anvil is removed, leaving the element installed in the orifice and the upper and lower walls of the body structure (between the orifice and the open end of the body structure) sealed to each other.

Other embodiments of the invention are a method and apparatus for installing a C-shaped, tined anastomosis element around the open end of a tubular body structure (e.g., a graft vessel having an open end). The element is slid onto the vessel near its open end, the element's open ends are squeezed together (to change the element from a C-shaped to an O-shaped element), an anvil is inserted in the open end of the vessel, and the element is advanced toward the vessel's open end (causing the tines to pierce the vessel tissue around the open end and evert the tissue at the open end as they curl against the anvil.

### BRIEF DESCRIPTION OF THE DRAWINGS

FIG. 1 is a cross-sectional view of a conventional anastomosis using rings to achieve side-to-side connection of blood vessels.

FIG. 2 is a cross-sectional view of a conventional anastomosis using a ring to achieve end-to-end connection of blood vessels.

FIG. 3 is a perspective view of a ring for use in performing anastomosis without hand sutures (with its tines in their initial, straight configuration).

FIG. 4 is a perspective view of the ring of FIG. 3, after its tines have been curled into their bent configuration.

FIG. 5 is a perspective view of the ring of FIG. 4, showing the manner in which the action of curling the tines everts the tissue (near the edges of an incision in the side wall of a blood vessel) to expose the intima of the vessel walls, during attachment of the ring to the vessel.

FIG. 6 is a top elevational view of another ring for use in performing anastomosis.

FIG. 7 is a top elevational view of a preferred embodiment of the inventive anastomosis element (for use in performing anastomosis).

FIG. 7A is a perspective view of element 20 of FIG. 7, with its tines bent into a configuration for installation at a vessel.

FIG. 8 is a perspective view of a portion of an embodiment of the inventive installation tool (for installing an anastomosis element), with element 20 of FIG. 7.

FIG. 8A is a perspective view of a variation on the anvil portion shown in FIG. 8.

FIG. 9 is a view, partially elevational and partially cross-sectional, of the anvil portion of the FIG. 8 tool, with element 20 of FIG. 7 and a graft vessel 30.

FIG. 10 is a perspective view of a portion of an embodiment of the inventive installation tool (for installing an anastomosis element).

FIG. 11 is an exploded perspective view of the installation tool of FIG. 10 with element 20 of FIG. 7.

FIG. 12 is another exploded perspective view of the installation tool of FIG. 10.

FIG. 12A is a perspective view of a portion of one of tine shields 12 of FIG. 12, when positioned to shield element 20.

FIG. 12B is a perspective view of a preferred implementation of element 25E of FIG. 12.

FIG. 12C is cross-sectional view of element 25E of FIG. 12B, taken along line 99-99 of FIG. 12B.

FIG. 13 is a perspective view of another embodiment of the inventive anastomosis element.

FIG. 14 is a side cross-sectional view of a first step of installation of the FIG. 13 element at the open end of a vessel.

FIG. 15 is a side cross-sectional view of a second step of installing the FIG. 13 element in the vessel.

FIG. 16 is a side cross-sectional view of a third step of installing the FIG. 13 element in the vessel.

FIG. 17 is a side cross-sectional view of a fourth step of installing the FIG. 13 element in the vessel.

FIG. 18 is a side cross-sectional view of a fifth step of installing the FIG. 13 element in the vessel.

FIG. 19 is a side cross-sectional view of the FIG. 13 element fully installed in the vessel.

FIG. 20 is a perspective view of the FIG. 13 element fully installed in the vessel.

FIG. 21 is a perspective view of a first step of installation of an anastomosis element at the open end of a vessel in accordance with the invention.

FIG. 22 is a perspective view of a second step of the installation process whose first step is shown in FIG. 21.

FIG. 23 is a side cross-sectional view of the step shown in FIG. 21.

FIG. 24 is a side cross-sectional view of the step shown in FIG. 22 (also showing the anastomosis element).

FIG. 25 is a side cross-sectional view of a third step of the installation process whose first step is shown in FIGS. 21 and 23.

FIG. 26 is a side cross-sectional view of the element of FIG. 24 fully installed in the vessel of FIG. 24.

FIG. 27 is a perspective view of a two-piece anvil designed in accordance with the invention.

FIG. 28 is a perspective view of the two-piece anvil of FIG. 27 being used to install an anastomosis element in a vessel.

FIG. 29 is a side cross-sectional the element of FIG. 28 when it has been fully installed in the vessel.

FIG. 30 is a perspective view of an embodiment of a C-shaped anastomosis element designed in accordance with the invention.

FIG. 31 is an end view of an anastomosis element (having the FIG. 30 design) that has been slid onto the open end of a blood vessel.

FIG. 32 is an end view of the element and vessel of FIG. 31 after the element has been deformed from a C-shape to an O-shape.

FIG. 33 is a side cross-sectional view of the element and vessel of FIG. 32, with an anvil inserted into the vessel's open end.

FIG. 34 is a perspective view of the element and vessel of FIG. 31, after the element has been fully installed at the vessel's open end and the tissue around the open end has been everted.

FIG. 35 is a perspective view of a portion of an embodiment of the installation tool that includes retractable hooks for use in positioning the end of the vessel over the anvil.

FIG. 36 is a side cross-sectional view of an embodiment of the inventive anvil which has holes and a central channel formed therein for applying suction to a vessel that is being (or has been) positioned on the anvil.

FIG. 37 is a perspective view of the anvil of FIG. 36.

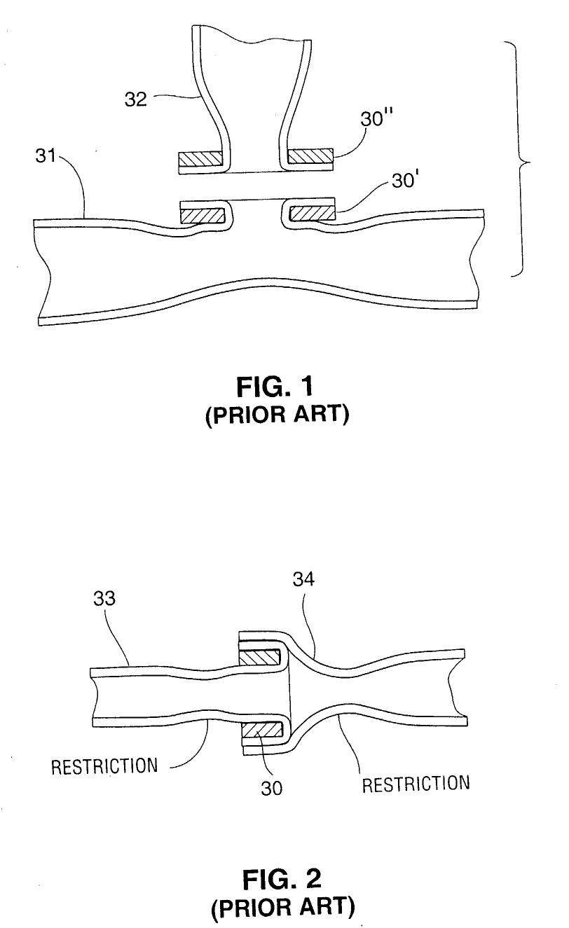

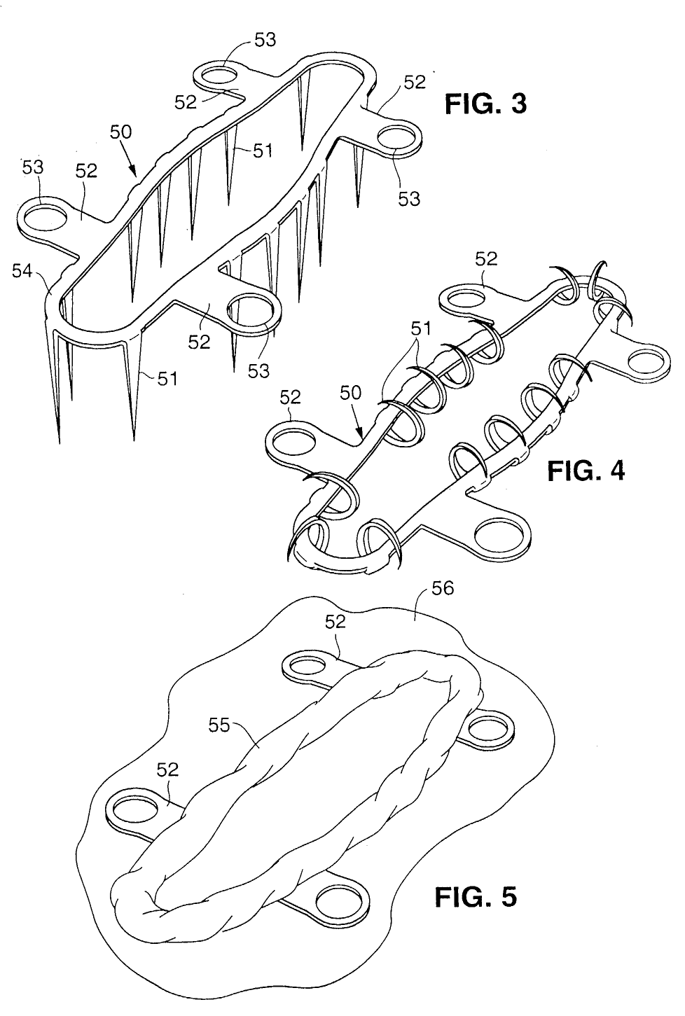

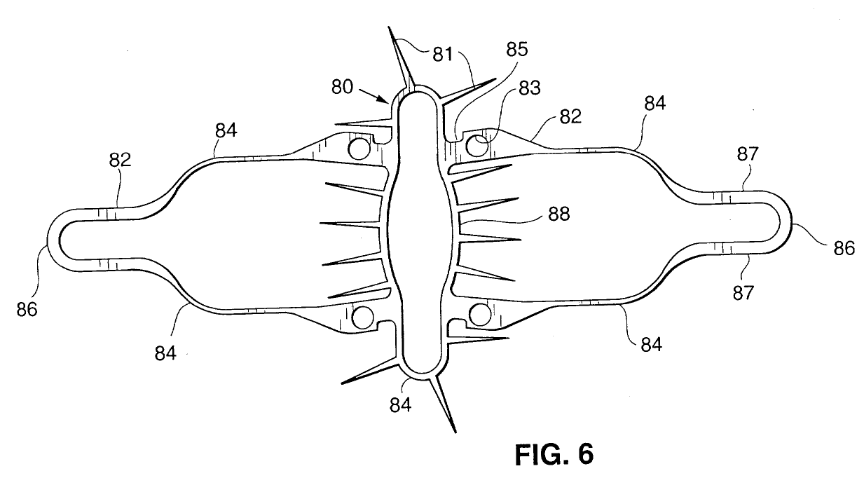

### DETAILED DESCRIPTION OF THE PREFERRED EMBODIMENTS

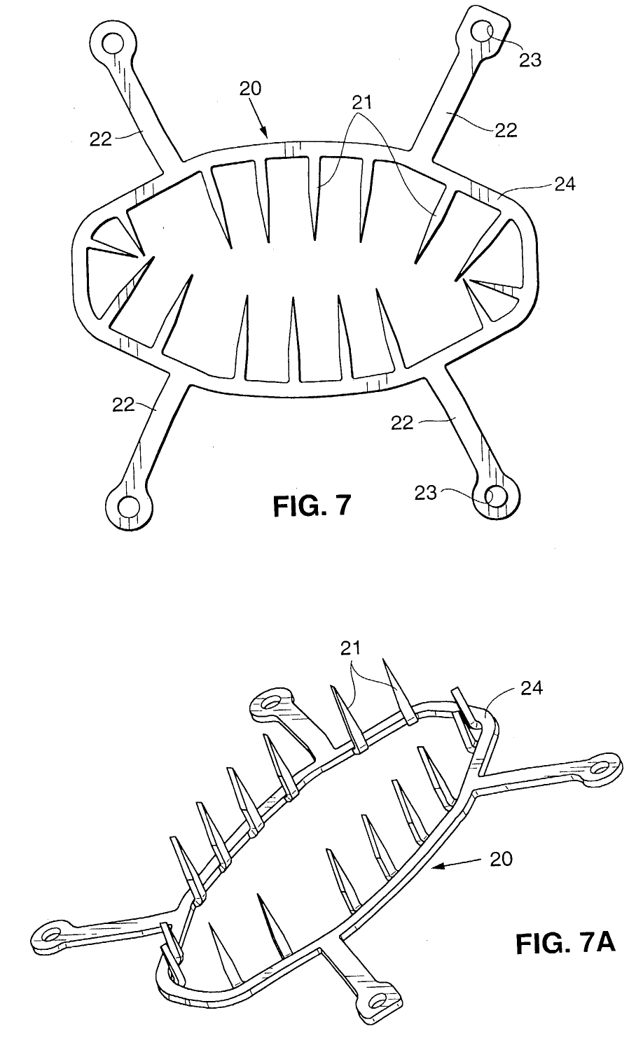

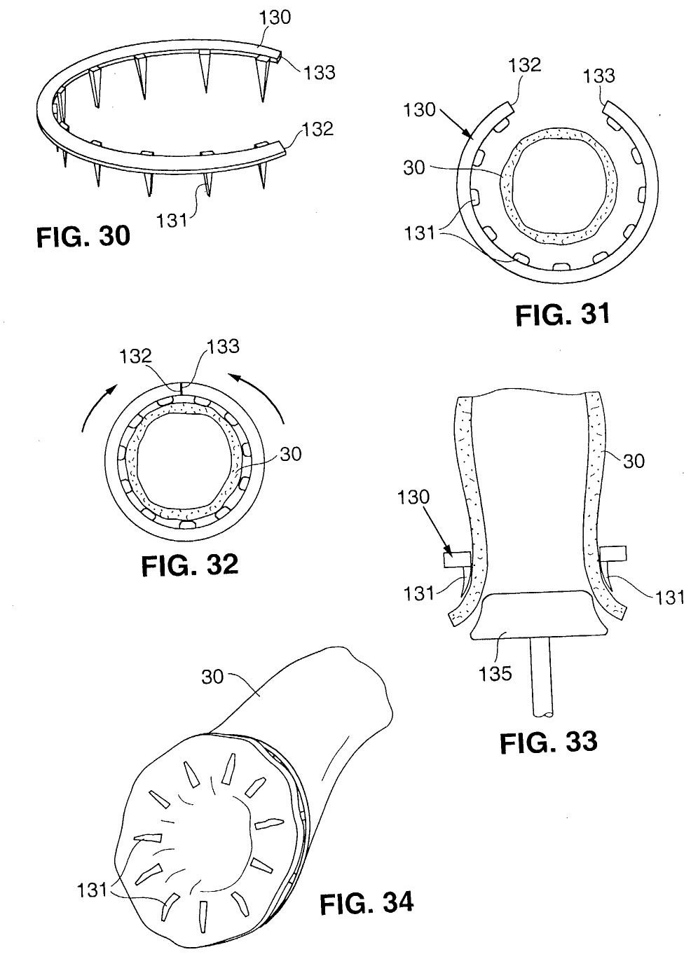

Throughout the disclosure, including in the claims, the term “element” used with reference to an anastomosis element embodying the invention (or used in performing an embodiment of the invention) denotes an object defining a central orifice, and having a central portion (at least partially surrounding the central orifice) from which deformable tines protrude. The element can be closed (in the sense that the central portion totally surrounds the central orifice, as does central ring portion 24 of element 20 of FIG. 7) or it can be open (in the sense that the central portion defines and partially surrounds the central orifice, as does the C-shaped ring portion of element 130 of FIG. 30). Also, the element can be manufactured in a flat configuration (e.g., stamped from a flat sheet of metal) or a configuration that is not flat (e.g., so as to have a cylindrical central portion, or other three-dimensional central portion).

Throughout the disclosure, including in the claims, the term “tine” used in a broad sense to denote any protrusion from the central portion of an anastomosis element which is capable of penetrating the tissue of a body structure to enable the anastomosis element to be installed in the body structure. A “tine” can have either a pointed or non-pointed tip, and it can have any cross-section provided that is capable of penetrating the tissue of the body structure to enable the anastomosis element to be installed therein.

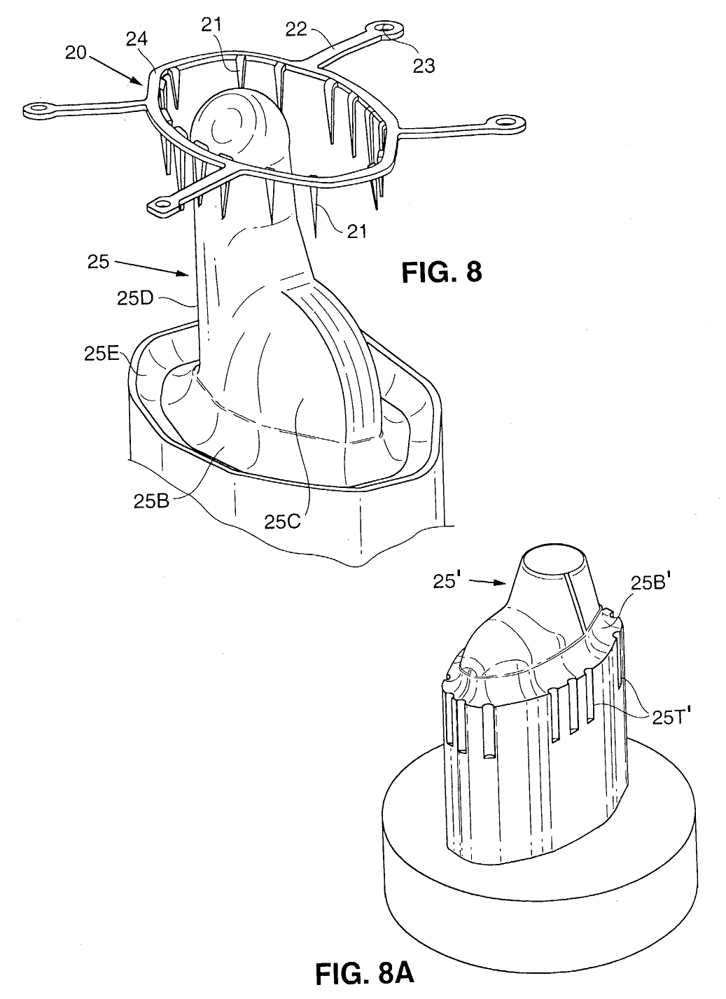

A preferred embodiment of the inventive anastomosis element will be described with reference to FIGS. 7 and 7A. A preferred embodiment of the installation tool of the invention, for installing such an element at the open end of a blood vessel will be described with reference to FIGS. 8, 9, 10, 11, and 12.

Element 20 (best shown in FIGS. 7 and 7A, and also shown in FIG. 8) is a preferred embodiment of the inventive anastomosis element. Element 20 has a central portion 24 in the form of a generally elliptical ring, four docking arms 22 that extend from the outer edge of ring portion 24, and a plurality of tines 21 that extend from the inner edge of ring portion 24. Each docking arm 22 defines a hole 23 for use in temporarily mounting element 20 to an installation tool, aligning element 20 with another identical element, and attaching the two aligned elements together. Element 20 is integrally formed from metal. Ring portion 24, tines 21, and arms 22 are deformable in the sense that once deformed from a first shape into a second shape, they will not relax back into the first shape from the second shape. Alternatively (as where the element is for use for a specific application known in advance), central portion 24 is not deformable and is instead manufactured with a desired (fixed) shape. Element 20 is typically manufactured in a flat configuration (as shown in FIG. 7) with tines 21 in the plane of central portion 24 and arms 22, such as by chemically etching (or photo etching) thin (e.g., 0.005 inch) sheet stock of stainless steel (and then additionally chemically etching, or photo etching, the tines to reduce the thickness of each tine to about 0.0004 inch). After being manufactured in a flat configuration, tines 21 are bent into a configuration in which they are perpendicular (or substantially perpendicular) to the plane of ring 24 as shown in FIG. 7A. Typically, tines 21 are about 0.055 inch in length, except for those at the ends of central portion 24 which are somewhat shorter (e.g., 0.040 inch in length) for ease in manufacture. By implementing the inventive anastomosis element with various tine layouts (including asymmetric tine layouts) the element can be implemented with tines which are all of equal length, or some or all of the tines can be longer relative to the central portion than are the tines shown in FIG. 7. Each of such implementations of the element can be inexpensively manufactured.

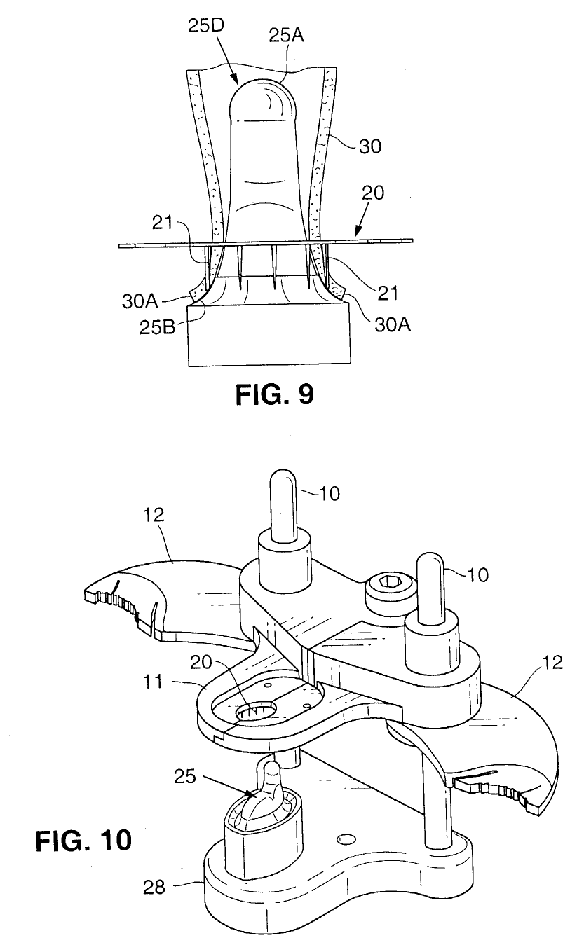

The installation apparatus shown in FIGS. 8-12 can be used to install element 20 (or another anastomosis element) at the open end of a vessel (e.g., at open end 30A of blood vessel 30 shown in FIG. 9). The installation apparatus includes base 28, guide pins 10 and anvil 25 (including outer tine track portion 25E of anvil 25) fixedly mounted to base 28, carriage 11 (including portions 11A, 11B, 11C, and 11D) slidably and rotatably mounted on pins 10, and tine shields 12 rotatably mounted around bushing portions 11D of carriage 11. Guide pins 10 extend up from base 28, and one bushing 11D (preferably made of bronze) is fitted around each pin 10. Preferably, there is sufficient friction between each pin 10 and the bushing 11D fitted around it so that the bushing remains in a fixed position along the pin unless manually pushed upward or downward along the pin. Preferably, such manual force is exerted by the user on the shelf portion 11E of each bushing 11D.

In all embodiments of the tool, the main function of the carriage (sometimes referred to as a “hammer”) is to drive the tines of the element through the vessel side wall into the anvil. After the vessel has been threaded through the element (and installation of the element to the vessel), the carriage must allow the vessel (with element) to be removed from the tool. Preferably (as in the embodiment of FIGS. 8-12), the carriage is hinged so that it can be moved from a closed configuration (for installation of the element) to an open configuration allowing removal of the vessel with element.

One of the shields 12 is fitted around each bushing 11D so as to rest on shelf portion 11E and to be rotatable relative to the bushing. Right portion 11A of carriage 11 is fitted around one bushing 11D so as to rest on one of shields 12 and to be rotatable relative to the bushing, and left portion 11B of carriage 11 is fitted around the other bushing 11D so as to rest on the other one of shields 12 and to be rotatable relative to the other bushing. Left and right portions 11A and 11B are prevented from pivoting relative to each other by inserting precision shoulder bolt 11C through aligned holes in portions 11A and 11B. At appropriate times during operation of the installation tool, bolt 11C is removed from portions 11A and 11B to allow the user to rotate portion 11A (about one pin 10) away from portion 11B (e.g., to allow removal of a graft vessel from the installation tool).

Carriage 11 is free to translate up and down relative to base 28 as pins 10 prevent sideways motion of carriage 11 relative to base 28. Anvil 25 has a central portion 25D (which defines vessel guide surface 25A, tine deforming concave surface 25B, and vessel shaping convex surface 25C) and an outer tine track portion 25E. To assemble the tool, portion 25E is placed above base 28, central portion 25D is inserted through hole 28A in base 28 and through the central channel of portion 25E, and portions 25E and 25D lock together. Surface 25B preferably has grooves for guiding tines 21 along radial paths as the tines begin to curl radially outward (as they are forced by the carriage against surface 25B). Portion 25E has a tine-deforming concave surface which meets surface 25B and guides each tine 21 along a subsequent portion of its curling path, to ensure that tines 21 curl enough to fasten element 20 securely to the vessel and evert the vessel tissue around element 20. Preferably, the tine-deforming surface of portion 25E has grooves (matching the grooves of surface 25B) for guiding tines 21 along their curling path. Preferably, portions 25E and 25D have grooves and ridges which fit together during assembly of the tool to lock together portions 25E and 25D with the proper relative orientation.

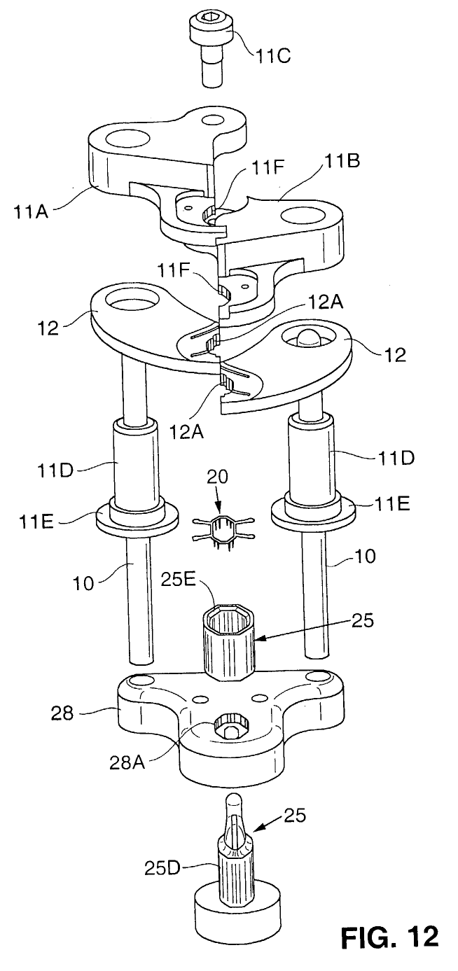

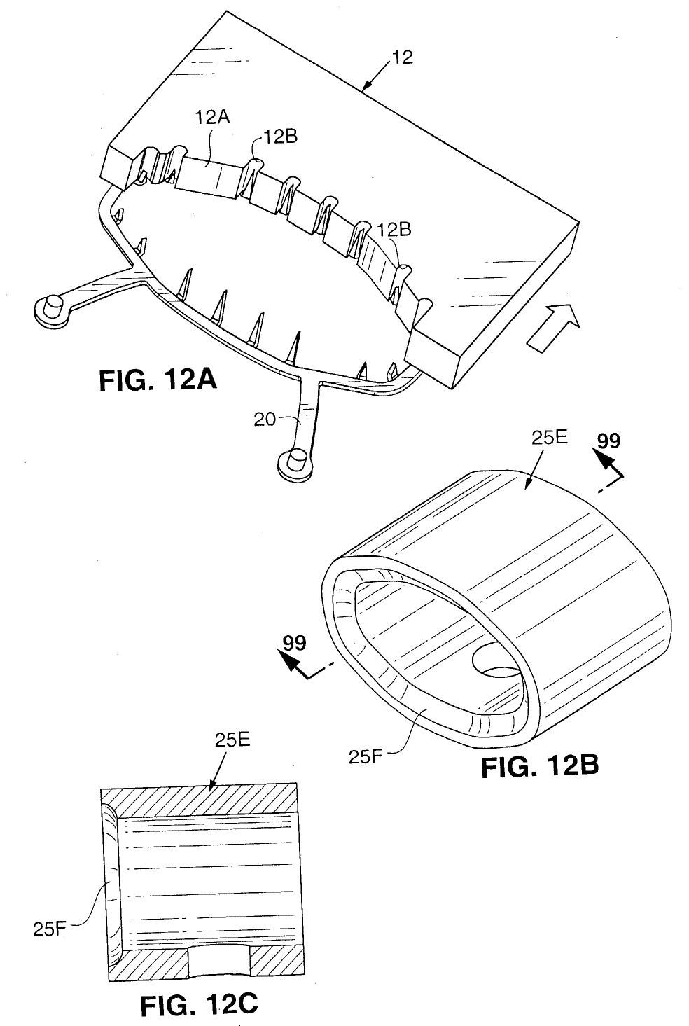

A preferred implementation of outer tine track portion 25E of anvil 25 (of FIG. 12) will be described with reference to FIGS. 12B and 12C. The FIG. 12B implementation of track portion 25E is milled from stainless steel. Tine curling surface 25F of track portion 25E is concave. The radius of curvature of surface 25F and the radius of curvature of surface 25B of anvil 25 are selected to ensure that tines 21 curl enough to fasten element 20 securely to the vessel and evert the vessel tissue around element 20. If portion 25E were omitted from the tool, tines 21 would curl radially outward (as they are forced by the carriage against surface 25B) but typically would not curl enough to both fasten element 20 securely to the vessel and evert the vessel tissue around element 20. With portion 25E installed around anvil portion 25D so that surface 25F meets surface 25B, tines 21 curl radially outward (when forced by the carriage against surface 25B) and then curl further radially outward until their tips point upward back up toward the carriage (when the tines are forced by the carriage against surface 25E). This ensures that tines 21 curl enough to fasten element 20 securely to the vessel and evert the vessel tissue around element 20. With tines 21 having length 0.057 inch, the radius of curvature of surface 25B would typically be 0.03125 inch, and the radius of curvature of surface 25E would typically be 0.010 inch. The radius of curvature of surface 25E should be sufficiently small to cause anvil 25 to curl tines 21 enough so that the curled tines securely fasten element 20 securely to the vessel and evert the vessel tissue around element 20. If the radius of curvature of surface 25E is too large, anvil 25 will curl tines 21 so that the tines' tips move radially outward only; not outward and then upward toward the carriage.

Anvil 25′ of FIG. 8A is a variation on anvil portion 25D of FIGS. 8 and 12. Tine deforming. concave surface 25B′ of anvil 25′ is designed for use with an outer tine track portion (not shown in FIG. 8A, but corresponding functionally to tine track portion 25E of FIG. 12). Grooves 25T′ are formed in the outer side wall of anvil 25′ for mating with corresponding tongues that protrude from the outer tine track portion, to attach anvil 25′ to the outer tine track portion with the correct relative orientation during assembly of the installation tool.

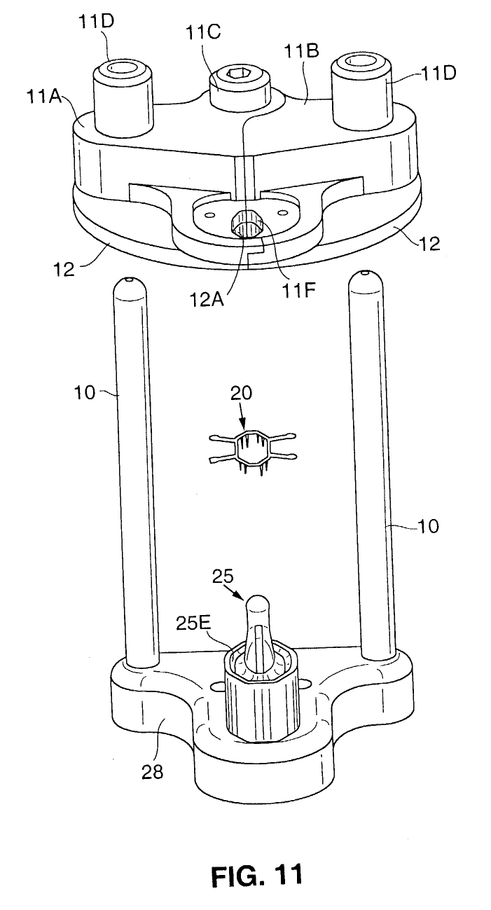

In use, the installation tool is assembled as shown in FIG. 11 (with surfaces 11F of portions 11A and 11B aligned with each other to define a first central orifice). Shields 12 are separated from each other (by rotating them about pins 10) to expose the bottom (distal) surface of the aligned portions 11A and 11B. The bottom surface of aligned portions 11A and 11B has four pins (not visible in FIG. 11) positioned for insertion through holes 23 of element 20. Element 20 is temporarily mounted to carriage 11 by insertion of these four pins through holes 23. Shields 12 are then rotated together, thus bringing together inner surfaces 12A of shields 12 to define a second central orifice, which is aligned with the first central orifice and with central portion 24 of element 20).

Then, a blood vessel which has been prepared in the standard way for anastomosis (e.g., vessel 30 of FIG. 9) is threaded (in the distal direction) through the central portion 24 (and aligned first and second central orifices). The vessel can be threaded through central portion 24 before or after bushings 11D of carriage 11 are inserted onto guide pins 10. When the vessel has been threaded through central portion 24 and bushings 11D have been inserted onto pins 10, the vessel is translated toward anvil 25 until the vessel's open end (e.g., end 30A of vessel 30) reaches surface 25B of anvil 25 and guide surface 25A extends into the vessel's interior. During positioning of the blood vessel (movement of the vessel through the aligned first and second central orifices into engagement with anvil 25), the aligned surfaces 12A shield the tips of tines 21 in order to prevent the tines from engaging and sticking the blood vessel.

In a preferred implementation, the tool includes a feature for locking together shields 12 to prevent them from rotating away from each other until the user unlocks them (following positioning of the vessel relative to the anvil but prior to piercing of the vessel by the tines of the element).

When shields 12 have been rotated together (as shown in FIG. 11) with surfaces 12A aligned with each other, the aligned surfaces 12A preferably define pockets (or grooves), such as grooves 12B shown in FIG. 12A, which are positioned to receive the tines of element 20, each pocket (or groove) being positioned to receive one of the tines. With surfaces 11F and 12A aligned with each other as in FIG. 11, shields 12 also retain element 20 in place against portions 11A and 11B, preventing the element from being dislodged during positioning of the vessel through portion 24 of element 20. When the vessel has been positioned through central portion 24 with its open distal end engaged with anvil surface 25B, shields 12 are separated from each other to expose the vessel to tines 21.

When the vessel has been positioned, the user lowers carriage 11 (along pins 10) relative to base 28, until the carriage forces tines 21 of element 20 to penetrate the vessel's side wall (near the vessel's distal end) and to curl radially outward against surface 25B (and the matching concave surface of portion 25E) of anvil 25. When viewed as in FIG. 9, surface 25B curls the left tines (e.g., the tine 21 at the left side of FIG. 9) radially toward the left side of FIG. 9, surface 25B curls the right tines (e.g., the tine 21 at the right side of FIG. 9 radially toward the right side of FIG. 9), and the act of curling tines 21 radially outward everts the tissue around the vessel's open end 30A to expose the intima of the vessel wall. While carriage 11 is lowered to curl tines 21, the end portion of vessel 30 is trumpeted or “spatulated” (spread and stretched) gently as carriage 11 and element 20 press the vessel's end portion over surfaces 25B and 25C of anvil 25. Vessel shaping convex surface 25C gently forms the end portion of vessel 30 into a hooded shape (resembling a cobra hood) suitable for creating a patent anastomotic junction.

After installation of element 20 at the open end of vessel 30 (and eversion of the tissue at the vessel's open end), carriage 11 is raised relative to base 28 (or base 28 is lowered away, or otherwise removed, from the vessel), bolt 11C is removed, and portions 11A and 11B are decoupled from each other and from vessel 30 and element 20.

More generally, in a class of embodiments of the inventive installation tool, an anastomosis element (having tines) is loaded on the carriage (hammer) with the tines facing away from the carriage surface. Then, tine shields are moved (preferably by rotating them) into place, effectively hiding the tines of the element. The carriage and anvil are separated from each other at this time. The graft vessel is then fed through the carriage and element, the carriage is aligned with the anvil, and the end portion of the vessel is placed over the anvil. Preferably the anvil has a vessel forming surface (shaped for forming the end portion of the vessel into a hooded shape) and the end portion of the vessel is placed over the anvil's vessel forming surface. The carriage is then advanced until the distal surfaces of the shields nearly touch the anvil. Then the shields are moved into an open position exposing the tines of the element, and the carriage is further advanced until the tines penetrate the vessel wall and bend outward (thereby installing the element, and everting the tissue, at the vessel's open end). The anvil is then removed from the vessel and carriage (or the vessel and carriage are removed from the anvil) and the element as decoupled from the carriage (e.g., by advancing the element off a set of pins which hold the element to the carriage). The carriage is then split or otherwise opened (e.g., by removing a locking element and pivoting or otherwise moving one part of the carriage relative to another part thereof) to release the vessel with installed element from the carriage. The result is an element installed at an open everted end of the vessel, with the end portion of the vessel formed into a shape (preferably a hooded shape) which will provide proper hemodynamics in the vessel following the anastomosis.

The vessel with installed anastomosis element is then moved to an anastomosis site at which a second anastomosis element has been installed in a second vessel (either in the second vessel's end in the case of an end-to-end anastomosis, or in an incision in the side wall of the second vessel in the case of an end-to-side anastomosis). The two elements are then aligned and fastened together to complete the anastomosis. Preferably, the two elements are pressed together and held together during the fastening process so as to seal the anastomosis, but optionally a sealant is used to provide a good fluid seal.

In some embodiments, the installation tool is implemented as a handheld device. Such a handheld device can be designed for one-handed or two-handed operation by the user, depending on the required level of vessel manipulation by the user. For example, the tool can be implemented with a vessel positioning feature or mechanism which eliminates or minimizes the need for vessel manipulation by the user (e.g., a vessel positioning subassembly employing vacuum, suction, or hooks to hold Temporarily and move the vessel).

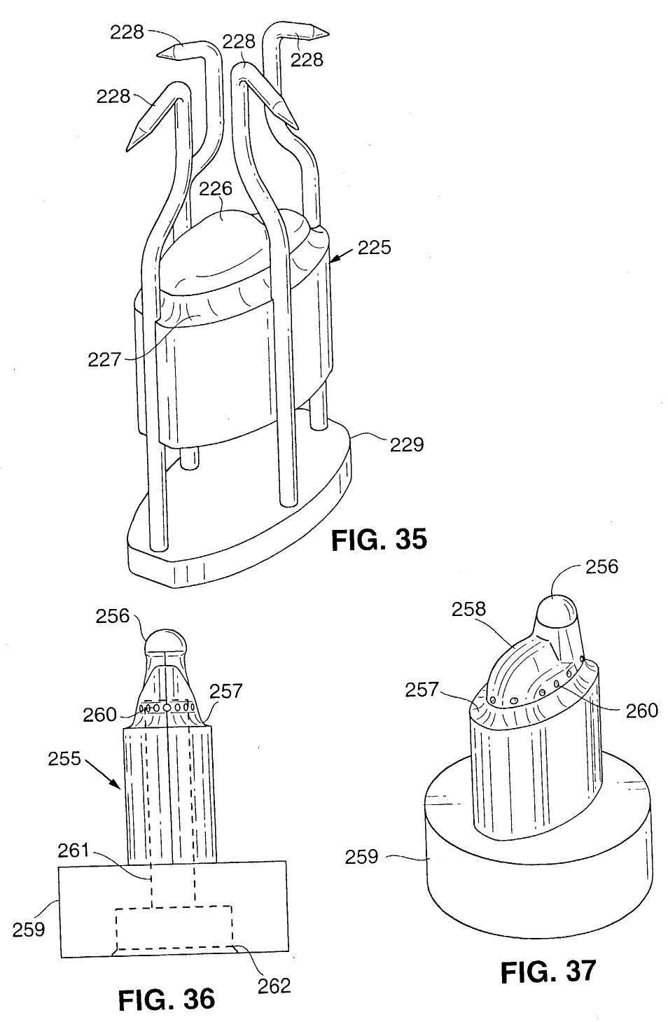

Next, with reference to FIG. 35, we describe an example of hooks (identified by reference numeral 228 in FIG. 35) for use in positioning a vessel's open end over the inventive anvil in a manner reducing the need for vessel manipulation by the user. Anvil 225 of FIG. 35, which performs the same function as anvil portion 25D of FIGS. 9 and 12 (except in that anvil 225 lacks an elongated vessel guide surface corresponding to surface 25A of FIG. 9), includes tine deforming concave surface 227, and vessel shaping convex surface 226. Four identical hooks 228 are translatably mounted at the sides of anvil 225. In preferred embodiments, each of hooks 228 is made of steel or NiTI alloy. Each hook 228 has a straight portion which is attached to element 229. Hooks 228 can be translated vertically relative to anvil 225 by moving element 229 upward or downward relative to anvil 225.

In operation of the FIG. 35 apparatus, hooks 228 would grip the vessel in such a manner that each hook's upper end penetrates the vessel sidewall near the vessel's open end (from the inside of the vessel to the outside). Then, hooks 228 are retracted (toward the bottom of FIG. 35) relative to anvil 225 to draw the vessel's end portion (near to the vessel's open end) over surfaces 226 and 227. After an anastomosis element has been installed in the vessel's end portion (e.g., in the manner described above with reference to FIGS. 8-12), the strip of vessel tissue between the element and the vessel's open end (including the tissue penetrated by hooks 228) is trimmed off to free the rest of the vessel (with installed element) from the installation tool. Preferably, a means (not shown) is provided for locking element 229 relative to anvil 225 during the element installation step and then unlocking element 229 after installation of the anastomosis element so that hooks 228 can be returned to their extended position.

Next, with reference to FIGS. 36 and 37, we describe anvil 255, which is another variation on anvil portion 25D of FIGS. 9 and 12, and which has holes extending therethrough for application of suction to a vessel to assist with positioning and shaping of the vessel during element installation. Anvil 255 of FIGS. 36 and 37 performs the same function as anvil portion 25D of FIGS. 9 and 12, and also defines a channel for applying suction to the vessel. Anvil 255 includes vessel guide surface 256, tine deforming concave surface 257, vessel shaping convex surface 258, and base 259. Holes 260 extend through surface 258, around the outer edge of surface 258 (where surface 258 meets surface 257). In one embodiment, twelve holes 260 are spaced around the outer edge of surface 258 (with reduced spacing between adjacent holes at the heel and toe ends of the anvil against which the tines at the heel and toe ends of the anastomosis element will be fired or otherwise pressed). To reduce the risk that vessel tissue will not cover all the holes 260 during vessel positioning and element installation (so that insufficient suction is applied to the vessel tissue), anvil 257 can be implemented with fewer than twelve holes 260. Channel 261 (shown in phantom view in FIG. 36) extends within anvil 255, from holes 260 to orifice 262 in the bottom of base 259. In operation of the apparatus of FIGS. 36 and 37, a vessel is positioned with its end portion (the vessel portion near to the vessel's open end) gently drawn over surfaces 257 and 258. Preferably, suction is applied (by a suction source not shown) through channel 261 and holes 260, to assist in guiding the vessel into the proper position (in which its end portion is drawn over surfaces 257 and 258), and during installation of an anastomosis element in the vessel's end portion (e.g., in the manner described above with reference to FIGS. 8-12) to secure and hold the vessel tissue to the anvil. After installation of the element in the vessel's end portion, application of the suction is terminated, to allow the vessel (with installed element) to be removed conveniently from the anvil.

Preferably, a luer fitting is press fit into channel 261 in base 259 and sealed in the proper position within base 259 (e.g., with Loctite sealant or another sealant). A suction line can be conveniently coupled to the luer fitting.

Anvil 225 can be made from stainless steel, with holes 260 and channel 261 machined therein. In one implementation, each hole 260 has a diameter of 0.0135 inch, base 259 has an outer diameter of 0.50 inch, orifice 262 has a diameter of 0.25 inch, and the length of channel 261 from the center of each hole 260 to the bottom of base 259 is 0.560 inch.

In some embodiments, the installation tool of the invention is equipped with a tactile or audible stop which indicates that the anastomosis element being installed has translated by the appropriate amount (relative to the anvil) to allow its tines to curl fully. In preferred embodiments, the installation tool is implemented as a handheld device which is designed to be conveniently operable by a surgeon in the operating room.

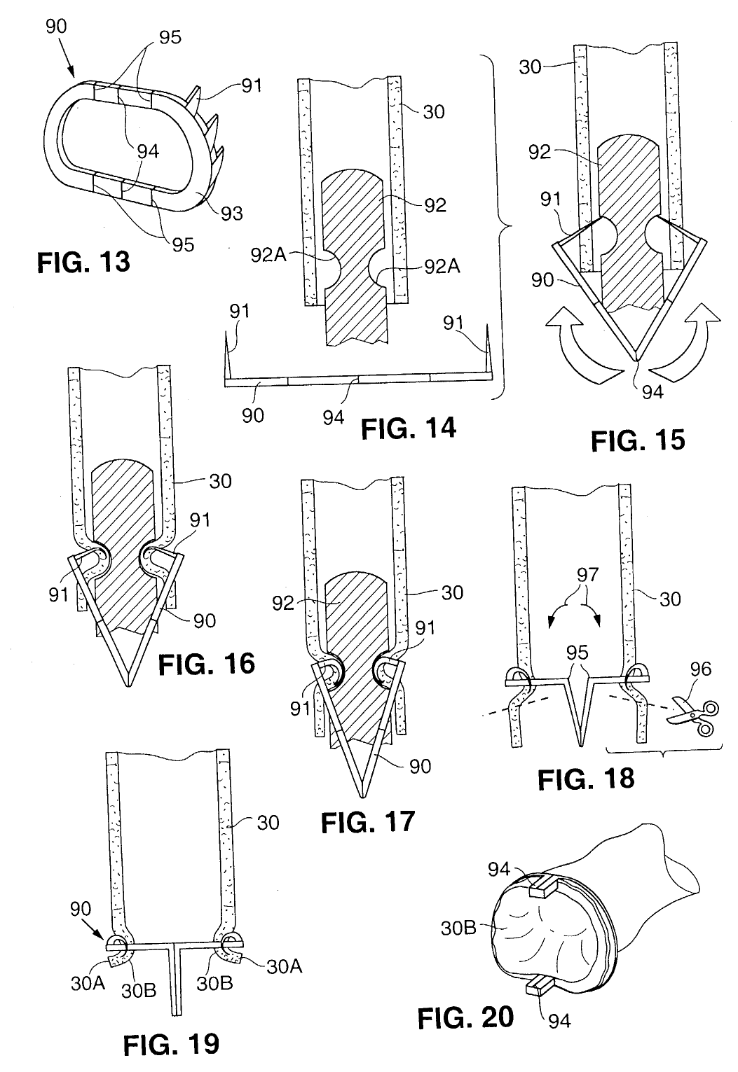

FIG. 13 is a perspective view of anastomosis element 90, which is another embodiment of the inventive anastomosis element for use in performing anastomosis without hand sutures. Element 90 is integrally formed from thin metal, is deformable, and includes a central portion 93 and tines 91 that extend from central portion 93. Central portion 93 is pre-folded, scored, or otherwise prepared for folding at central hinge portion 94 and side hinge portions 95.

To install element 90 at the open end of a tubular (e.g., cylindrical or generally cylindrical) body structure (such as blood vessel 30 of FIG. 14), an anvil 92 having tine deforming concave surfaces 92A is placed in the vessel's open end as shown in FIG. 14. Element 90 is aligned with its tines 91 parallel to, and central portion 93 coaxial with, the vessel's central longitudinal axis (as also shown in FIG. 14).

As shown in FIG. 15, the user then bends (folds) element 90 about central hinge portion 94 to cause tines 91 to pierce the vessel's side wall and engage surfaces 92A of anvil 92. In other words, the user exerts torque on central portion 93 tending to move the left end of the element (when viewed as in FIG. 15) and the right end of the element (when viewed as in FIG. 15) closer together, and central portion 93 folds along hinge portion 94 in response to this torque. The user continues to press together the two halves of folded element 90 to cause tines 91 to curl against surfaces 92A, as shown in FIG. 16, thereby everting the vessel tissue near the ends of tines 91. When tines 91 are fully curled against surfaces 92A, element 90 and vessel 30 have the configuration shown in FIG. 17.

Then, anvil 92 is removed from the vessel and the user pinches together the central portions of element 90 (those portions between hinge portion 94 and side hinge portions 95) as shown in FIG. 18. This causes element 90 to bend about hinge portions 95 as shown in FIG. 18, thereby further everting the vessel tissue near the ends of tines 91. Optionally, a force-exerting element or mechanism is provided for exerting force on the upper surface of element 90 (viewed as in FIG. 18) in the direction of arrows 97, to assist in flattening the outer parts of element 90 (the portions of central portion 93 outside hinge portions 95).

Typically, the user then trims the end of vessel 30 (as indicated by scissors 96 of FIG. 18) to define a new vessel end 30A (as shown in FIG. 19). Element 90 installed at new end 30A of vessel 30 everts the vessel tissue to expose the vessel's intima 30B near end 30A. Alternatively, the user does not define a new vessel end (e.g, by trimming the existing end of the vessel) after element 90 is bent about hinge portions 95, and the act of bending of element 90 about hinge portions 95 everts the tissue to expose the vessel's intima at the vessel's existing end.

FIG. 20 is a perspective view of the vessel with element 90 fully installed at its open distal end, showing the exposed folded hinge portions 94 of element 90 and the exposed intima 30B of the vessel. In the FIG. 20 configuration, the vessel is ready to be used to produce an end-to-side or end-to-end anastomosis.

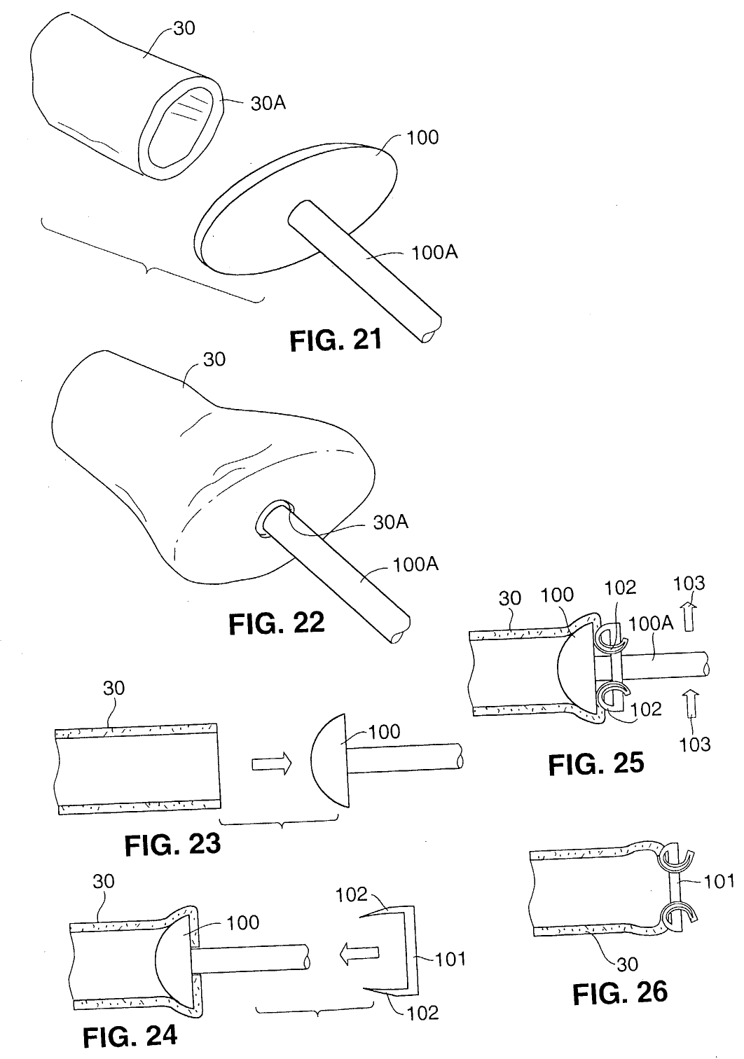

Another method and apparatus for installing an anastomosis element in the open distal end of a generally cylindrical body structure (such as blood vessel 30 of FIG. 21) in accordance with the invention will be described with reference to FIGS. 21-26. Anvil 100 (mounted at the distal end of elongated stem 100A) is advanced toward the open end 30A of vessel 30 (as shown in FIGS. 21 and 23) and is inserted into the open end 30A (as shown in FIGS. 22 and 24), causing the tissue around open end 30A to curl over the outer edge of the anvil's proximal surface as shown. Then, as shown in FIG. 24, anastomosis element 101 having plurality of tines 102 is advanced toward anvil 100. Element 101 is then installed by driving (or firing) it so that tines 102 penetrate the vessel tissue around end 30A and curl radially outward against anvil 100 into the configuration shown in FIG. 25,. The action of curling the tines everts the vessel tissue near to end 30A, exposing the vessel's intima. Stem 100A is then tilted in the direction of arrows 103 of FIG. 25, to reduce the cross-section of anvil 100 facing the installed element 101, and stem 100A is then retracted from the vessel to remove anvil 100 from within the vessel. As a result, element 101 is left installed in the end of vessel 30 as shown in FIG. 26, with the element everting the tissue near the vessel's end (to expose the vessel's intima so that the intima can be joined to tissue of another body structure). Vessel 30 with installed element 101 is ready to be used to effect an end-to-side or end-to-end anastomosis.

An installation apparatus of the type disclosed in above-referenced provisional application 60/152,001 can be used to advance and retract anvil 100, element 101, and components for driving element 101 in an appropriate sequence.

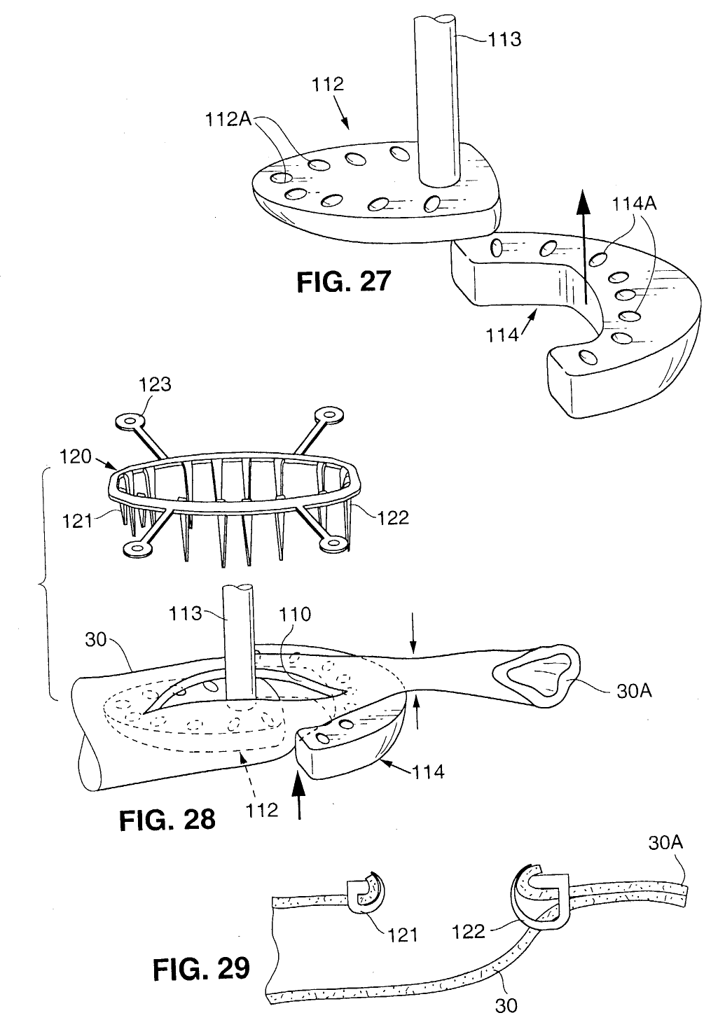

With reference to FIGS. 27-29, we next describe another method and apparatus for installing an anastomosis element at the site of an incision (incision 110) in the side wall of a vessel (blood vessel 30 shown in FIGS. 28 and 29), in such a manner that the installed element can be used to effect an end-to-end anastomosis (with an anastomosis element installed in the end of another vessel) or an end-to-side anastomosis (with an anastomosis element installed in the side of another vessel). A two-piece anvil (best shown in FIG. 27) is used to install the element. The two-piece anvil comprises intraluminal anvil 112 (mounted at the distal end of stem 113) and extracorporeal anvil 114. In use, anvil 112 is inserted downward through the incision (e.g., incision 110) into the vessel interior, as shown in FIG. 28. Anvil 114 is not inserted into the vessel interior, but is advanced upward into engagement with the outer sidewall of the vessel (as shown in FIG. 28) until the generally flat upper surface of anvil 112 (which defines tine-forming pockets 112A) is coplanar with the generally flat upper surface of anvil 114 (which defines tine-forming pockets 114A). To install anastomosis element 120 (or a similar tined anastomosis element) in the vessel, the element is advanced downward until tines 121 pierce the upper wall of the vessel (at locations surrounding incision 110 and above 112), tines 122 pierce both upper and lower walls of the vessel (at locations surrounding incision 110 and above anvil 114), and all the tines have curled radially outward in response to being forced against pockets 112A and 114A of the coplanar flat surfaces of anvils 112 and 114. After tines 121 and 122 are curled, anvil 114 is removed and stem 113 is manipulated to pull anvil 112 out from the vessel through incision 110. As shown in FIG. 29, when element 120 is fully installed in the vessel, its curled tines 121 and 122 evert the vessel tissue around the incision (exposing the intima so that the intima can be joined to tissue of another body structure).

In order to install element 120 in vessel 30 in such a manner that the installed element can be used to effect an end-to-end or end-to-side anastomosis (i.e., connected with another element installed in a second vessel, either in the second vessel's end in the case of an end-to-end anastomosis, or in an incision in the second vessel's side wall in the case of an end-to-side anastomosis), element 120 preferably seals the open end 30A of the vessel as shown in FIG. 29. Docking arms 123 of element 120 are used to grasp the installed element so that it can be deformed to control the size and shape of the orifice in which it is installed, to align the installed element with another anastomosis element installed in a second vessel, and to connect the two installed elements together to effect an anastomosis. We define the end of incision 110 nearest to open end 30A as the “toe” of the incision, and the end of incision farthest from open end 30A as the “heel” of the incision.

As shown in FIG. 28, tines 121 at the “heel” end of element 120 (to be installed at the heel end of incision 110) as well as the tines along the element's sides between the element's toe and heel ends, are preferably shorter than tines 122 at the “toe” end of element 120 (to be installed at the toe end of incision 110). Tines 122 are preferably sufficiently long to pierce both the top and bottom side walls of vessel 30 at the incision's toe end (before they have been curled) and then to pierce both top and bottom walls a second time when being curled (in response to force exerted thereon by anvil 114), so that the curled tines 122 press together the top and bottom side walls to seal the vessels's open end 30A. Thus, the open end 30A is sealed as part of the same operation which installs element 120. Also during such operation, the force exerted by anvils 112 and 114 on the vessel tissue between them forms the vessel into an ideal “hooded” shape for effecting a patent anastomosis.

As shown, intraluminal anvil 112 has a convex, body structure-engaging surface and extracorporeal anvil 114 has a concave (U-shaped), body structure-engaging surface shaped to mate with the convex, body structure-engaging surface of the intraluminal anvil with organ tissue pinched between the two body structure-engaging surfaces.

Typically, incision 110 is a longitudinal incision approximately 1.5 mm to 2 mm in length.

It is contemplated that a hand-held installation instrument would be used to install element 120 as described with reference to FIGS. 27-29. The instrument would include a feature or mechanism for controlling the relative spacing of anvils 112 and 114, as well as a mechanism for firing the element against the aligned anvils. The instrument would be capable of initially separating anvil 114 from anvil 112 to allow insertion of a transverse section of a graft vessel (having a pre-cut arteriotomy) therebetween, then moving the anvils together to pinch the vessel between them while the anastomosis element is fired, and finally separating the anvils from each other to allow removal of the vessel (in which the element has been installed).

Another class of embodiments of the invention will be described with reference to FIGS. 30-34. These embodiments employ a C-shaped, tined anastomosis element, such as element 130 of FIG. 30. Element 130 has a deformable, C-shaped body with open ends 132 and 133, and tines 131 extending at least substantially perpendicular to the plane of the C-shaped body. To install element 130 around the open end of a cylindrical body structure (e.g., graft vessel 30 of FIGS. 31-34), element 130 is slid sidewise (toward the top of FIG. 31) around the vessel a short distance above the vessel's open end, with tines 131 pointing toward the open end (as shown in FIG. 31). Open ends 132 and 133 of element 130 are then squeezed together (to change the element from a C-shaped to an O-shaped element that is present around the entire circumference of vessel 30) as shown in FIG. 32. Then, an anvil 135 is inserted into the vessel's open end as shown in FIG. 33 (thereby flaring the end portion of the vessel), and element 130 is advanced (downward when viewed as in FIG. 33) toward the vessel's open end, causing tines 131 to pierce the vessel tissue around the open end and curl radially outward (away from each other) against the anvil, thereby everting the vessel tissue at the open end as shown in FIG. 34. Anvil 135 is then removed from the vessel, leaving element 130 installed at the vessel's everted, open end. The embodiment of FIGS. 30-34 is expected to be useful to eliminate the need for extensive skeletonization of an artery (such as the internal mammary artery) during bypass surgery. The C-shape of the element's body eliminates the need to thread a graft vessel through the element, and there is no significant risk that the tines will get caught on the adventitia (outer layer) of the graft vessel. It also allows for a more intuitive way of loading the graft vessel into the element-firing device, with the added benefit of potentially reducing vessel manipulation, which in a vessel as important as the internal mammary artery is a significant advantage. It should be understood that while certain forms of the present invention have been illustrated and described herein, the invention is not to be limited to the specific forms or arrangements of parts described and shown or the specific methods described.

What is claimed is:

1. An anastomosis element, comprising:
a central portion that extends at least partially around a central orifice, the central portion having an inner edge that faces the central orifice; and
deformable tines extending from the inner edge of the central portion.

2. The element of claim 1, wherein the element is integrally formed from metal.

3. The element of claim 1, wherein the central portion has a heel end and a toe end, the tines include a first set of tines at the heel end and a second set of tines at the toe end, and each of the tines in the second set is longer than each of the tines in the first set.

4. The element of claim 1, wherein the central portion is a ring portion that extends completely around the central orifice.

5. An anastomosis element, comprising:
a central portion extending at least partially around a central orifice and having a first end and a second end; and
deformable tines extending from the central portion,
wherein the central portion has a central hinge portion along which the central portion folds in response to torque, exerted on the central portion, tending to move the first end and the second end closer together.

6. The element of claim 5, wherein the central portion also has a side hinge portion between the central hinge portion and the first end, and a second side hinge portion between the central hinge portion and the second end.

7. The element of claim 6, wherein the side hinge portion and the second side hinge portion are configured so that when the central portion has been folded about the central hinge portion and the tines installed in the side wall of a tubular body structure, the central portion folds along the side hinge portion and the second side hinge in response to pinching together of a first part of the central portion between the central hinge portion and the side hinge portion and a second part of the central portion between the central hinge portion and the second side hinge portion.

8. The element of claim 5, wherein the element is integrally formed from metal.

9. An anastomosis element, comprising:
a C-shaped, deformable central portion; and
deformable tines extending from the central portion.

10. The element of claim 9, wherein the element is integrally formed from metal.

11. A method for installing an anastomosis element in a body structure having a tubular end portion defining an open end, wherein the element has a central portion and deformable tines extending from the central portion, said method including the steps of:
(a) removably mounting the element to a carriage assembly such that the tines are shielded from the body structure;
(b) passing the end portion of the body structure through the central portion and positioning the open end of the body structure against an anvil;
(c) exposing the tines to the end portion of the body structure and actuating the carriage assembly to drive the tines against the anvil, thereby causing the tines to pierce the body structure and curl radially outward from the central portion to evert tissue of the body structure around the open end.

12. The method of claim 11, wherein the body structure is a blood vessel having an intima, and step (c) leaves the element installed in the vessel with the tines everting said tissue so as to expose the intima around the open end.

13. The method of claim 12, wherein the anvil has a convex shaping surface, step (b) includes the step of positioning the body structure's end portion over the shaping surface, and during step (c) the vessel's end portion is formed into a hooded shape in response to force exerted thereon by the element and the anvil's shaping surface.

14. The method of claim 11, wherein the anvil has a protruding guide portion and wherein step (b) includes the step of positioning the body structure so that the guide portion of the anvil extends inside the body structure's end portion.

15. A method for installing an anastomosis element in a body structure having a tubular end portion defining an open end, wherein the element has a central portion and deformable tines extending from the central portion, said method including the steps of:
(a) inserting an anvil having a tine-curling surface into the open end of the body structure, so that tissue of the body structure's end portion covers the tine-curling surface;
(b) after step (a), driving the element against the tissue covering the tine-curling surface, thereby causing the tines to pierce said tissue and engage the anvil, and causing the tines to curl radially outward from the central portion against the anvil thereby everting said tissue; and
(c) after step (b), retracting the anvil from the body structure so that the element remains installed at the open end of the body structure.

16. The method of claim 15, wherein the body structure is a blood vessel.

17. A method for installing an anastomosis element in a body structure using a composite anvil comprising an intraluminal anvil having a first tine-forming surface and an extracorporeal anvil having a second tine-forming surface, wherein the body structure has a tubular end portion surrounding a lumen and defining an open end, and the element has a central portion with a heel end and a toe end and deformable tines extending from the central portion including the heel end and the toe end, said method including the steps of:
(a) inserting the intraluminal anvil into the lumen through an incision in the end portion, wherein the incision is spaced from the open end;
(b) advancing the extracorporeal anvil into engagement with an outer surface of the end portion without entering the lumen, until the first tine-forming surface is aligned with the second tine-forming surface, thereby forming an aligned anvil pair which pinches the body structure closed at a location between the incision and the open end; and
(c) after step (b), orienting the element with the toe end facing the open end and the heel end facing away from the open end, and advancing the element toward the aligned anvil pairs until the tines at the heel end pierce a first side of the end portion of the body structure, the tines at the toe end pierce both the first side of the end portion and a second side of the end portion opposite to the first side, and all the tines curl radially outward in response to being forced against the first tine-forming surface and the second tine-forming surface.

18. The method of claim 17, also including the step of:
(d) after step (c), removing the intraluminal anvil from the lumen through the incision, leaving the element installed in the body structure with the tines piercing and everting tissue of the body structure surrounding the incision, and sealing together the first side and the second side of the end portion at said location between the incision and the open end.

19. The method of claim 17, wherein the body structure is a blood vessel having an intima, and step (d) includes the step of leaving the element installed in the body structure with said tines everting said tissue so as to expose the intima around the incision.

20. A method for installing an anastomosis element in a body structure having a tubular portion surrounding a lumen and defining an open end, the element having a central portion with a first end and a second end and deformable tines extending from the central portion, said method including the steps of:
(a) inserting an anvil through the open end into the lumen;
(b) folding the central portion about a first axis between the first end and the second end, thus causing the tines to pierce the body structure and curl against the anvil;
(c) removing the anvil from the lumen through the open end; and
(d) after steps (b) and (c), folding the central portion along a second axis and a third axis by pinching together a first part of the central portion between the first axis and the second axis and a second part of the central portion between the first axis and the third axis.

21. The method of claim 20, also including the step of:
(e) cutting off an end portion of the tubular portion to expose a surface of the body structure as a new open end of the tubular portion, leaving the element installed in the body structure with the tines piercing and everting tissue of the body structure around the new open end.

22. The method of claim 20, wherein the body structure is a blood vessel having an intima, and step (e) includes the step of leaving the element installed in the body structure with said tines everting said tissue so as to expose the intima around the new open end.

23. A method for installing an anastomosis element in a body structure having a tubular portion surrounding a lumen and defining an open end, the element having a C-shaped, deformable central portion and deformable tines extending from the central portion, the central portion having a first open end and a second open end, said method including the steps of:
(a) sliding the element onto the tubular portion of the body structure;
(b) squeezing together the first open end of the element and the second open end of the element, to change the C-shaped, deformable central portion to an O-shaped central portion;
(c) inserting an anvil into the lumen through the open end of the body structure, thereby flaring the tubular portion of the body structure;
(d) after steps (b) and (c), advancing the element toward the open end to cause the tines to pierce the tubular portion of the body structure and curl against the anvil, so that the tines evert tissue of the body structure at the open end as a result of curling against the anvil.

24. The method of claim 23, wherein the body structure is a blood vessel having an intima, and also including the step of:
(e) after step (d), removing the anvil from the lumen through the open end, leaving the element installed in the blood vessel with the tines everting said tissue at the open end so as to expose the intima around the open end.

25. An apparatus for installing an anastomosis in a body structure having a tubular end portion defining an open end, wherein the element has a central portion and deformable tines extending from the central portion, said apparatus comprising:
an anvil assembly; and
a carriage assembly translatably mounted to the anvil assembly for translation toward the anvil assembly, wherein the carriage assembly is configured to support the element and to drive the element toward the anvil assembly when the carriage assembly is translated toward the anvil assembly, the carriage assembly defines an opening that is sized and shaped for guiding the tubular end portion of the body structure through the central portion into engagement with the anvil assembly, the carriage assembly includes a tine shield assembly having a first state and a second state, the tine shield assembly in the first state shields the tines of the element when said element is supported by the carriage assembly, and the tine shield assembly in the second state exposes the tines of the element when said element is supported by the carriage assembly.

26. The apparatus of claim 25, wherein the anvil assembly includes:
an anvil having a convex, body structure-shaping surface shaped to form the tubular end portion of the body structure into a hooded shape in response to actuation of the carriage assembly to drive the element against the tubular end portion and the anvil to press the tubular end portion against the body structure-shaping surface, when the tubular end portion has been inserted through the central portion and positioned in engagement with the anvil.

27. The apparatus of claim 26, wherein the anvil also has a concave, tine-curling surface around the body structure-shaping surface.

28. The apparatus of claim 27, wherein the anvil defines a channel, and at least one of the tine-curling surface and the body structure-shaping surface defines suction holes in fluid communication with the channel.

29. The apparatus of claim 27, wherein the base defines an anvil-mounting opening, and the anvil includes:
a central part defining the body structure-shaping surface and a radially inner portion of the tine-curling surface; and
an outer part defining a radially outer portion of the tine-curling surface and a mounting surface, wherein the outer part abuts a first side of the base and the central part extends through the anvil-mounting opening from a second side of the base into engagement with the mounting surface of the outer part.

30. The apparatus of claim 29, wherein the mounting surface of the outer part defines linear features, the central part has a side wall defining additional linear features, and the additional linear features are shaped to lock with the linear features of the outer part's mounting surface.

31. The apparatus of claim 25, wherein the anvil assembly includes a base and an anvil, the base defines an anvil-mounting opening, and the anvil has a concave, tine-curling surface, said anvil comprising:
a central part defining a radially inner portion of the tine-curling surface; and
an outer part defining a radially outer portion of the tine-curling surface and a mounting surface, wherein the outer part abuts a first side of the base and the central part extends through the anvil-mounting opening from a second side of the base into engagement with the mounting surface of the outer part.

32. The apparatus of claim 31, wherein the anvil mounting surface of the outer part defines linear features, the central part has a side wall defining additional linear features, and the additional linear features are shaped to lock with the linear features of the outer part's mounting surface.

33. The apparatus of claim 25, wherein the carriage assembly defines anvil receiving openings, and the anvil assembly includes:
a base;
carriage guide members extending from the base and configured to extend through the anvil receiving openings;
an anvil mounted to the base and having a convex, body structure-shaping surface shaped to form the tubular end portion of the body structure into a hooded shape in response to actuation of the carriage assembly to drive the element against the tubular end portion and the anvil to press the tubular end portion against the body structure-shaping surface, when the tubular end portion has been inserted through the central portion and positioned in engagement with the anvil.

34. The apparatus of claim 33, wherein the base defines an anvil-mounting opening, the anvil has a concave, tine-curling surface, and the anvil comprises:
a central part defining the body structure-shaping surface and a radially inner portion of the tine-curling surface; and
an outer part defining a radially outer portion of the tine-curling surface and a mounting surface, wherein the outer part abuts a first side of the base and the central part extends through the anvil-mounting opening from a second side of the base into engagement with the mounting surface of the outer part.

35. The apparatus of claim 25, wherein the anvil assembly includes an anvil, and wherein said apparatus also includes:
a body structure-pulling assembly translatably mounted to the anvil assembly, the body structure-pulling assembly being translatable between a first state and a second state, wherein the body structure-pulling assembly is configured to engage the tubular end portion of the body structure in the first state after the tubular end portion has been inserted through the central portion, and the body structure-pulling assembly is configured to pull the tubular end portion into engagement with the anvil as said body structure-pulling assembly translates from the first state to the second state.

36. The apparatus of claim 35, wherein the body structure-pulling assembly includes a set of hooks, each of the hooks having a straight portion which engages the anvil assembly and is translatable linearly relative to the anvil assembly.

37. The apparatus of claim 25, wherein the anvil assembly includes an anvil defining concave, tine-curling surface and convex, body structure-shaping surface, wherein the anvil defines a channel, and wherein at least one of the tine-curling surface and the body structure-shaping surface defines suction holes in fluid communication with the channel.

38. An apparatus for installing an anastomosis element in a body structure having a tubular end portion surrounding a lumen and defining an open end, wherein the element has a central portion and deformable tines extending from the central portion, said apparatus comprising:
a first part comprising a stem and an intraluminal anvil mounted to the stem, wherein the intraluminal anvil has a generally flat, intraluminal tine-forming surface, and the intraluminal anvil is sized and shaped for insertion into the lumen through an incision in the tubular end portion; and
a second part defining a generally flat, extracorporeal anvil surface, configured to be aligned with the intraluminal tine-forming surface.

39. The apparatus of claim 38, wherein each of the intraluminal tine-forming surface and the extracorporeal anvil surface defines tine-forming pockets.

40. The apparatus of claim 38, wherein the intraluminal anvil has a convex, body structure-engaging surface and the extracorporeal anvil has a concave, body structure-L.,gaging surface shaped to mate with the convex, body structure-engaging surface of the intraluminal anvil with body structure tissue pinched between said concave, body structure-engaging surface of the extracorporeal anvil and said convex, body structure-engaging surface of the intraluminal anvil.
